(19) United States
(12) Patent Application Publication
(10) Pub. No.: US 2004/0010180 A1
(43) Pub. Date: Jan. 15, 2004

(54) Cardiac assist system

(76) Inventor: Sean K. Scorvo, Bend, OR (US)

Correspondence Address: Chernoff Vilhauer McClung & Stenzel, L.L.P., 1600 ODS Tower, 601 SW Second Avenue, Portland, OR, 97204-3157, US

(21) Appl. No.: 10/439,927

(22) Filed: May 16, 2003

Related U.S. Application Data
(60) Provisional application No. 60/381,461, filed on May 16, 2002.
(60) Provisional application No. 60/386,118, filed on Jun. 3, 2002.

(51) Int. Cl. A61N 1/362

(52) U.S. Cl. 600/16

(57) ABSTRACT

A cardiac assist system for treating heart disease includes a cardiac assist device that restrains dilation of the heart and assists the heart's contraction by controlled actuation of contractile transducers engaging the heart's surface.

### CROSS-REFERENCE TO RELATED APPLICATIONS

This application claims the benefit of U.S. patent application Ser. No. 60/381,461 filed May 16, 2003 and the benefit of U.S. patent application Ser. No. 60/386,118 filed Jun. 3, 2002.

### BACKGROUND OF THE INVENTION

The present invention relates to a method and apparatus for treating cardiac disease and related valvular dysfunction, and, more particularly, to a cardiac assist system and method.

In the United States alone, about five million people suffer from congestive heart failure. In addition, about 400,000 new patients are diagnosed in the United States each year making congestive heart failure one of the most rapidly advancing diseases. Economic costs of the disease have been estimated at $38 billion annually. The causes of congestive heart failure are varied and not fully understood and, while a substantial effort has been made to develop treatments for the disease, the only permanent treatment presently available is a heart transplant. Heart transplant procedures are expensive, risky and extremely invasive, and a shortage of hearts donated for transplant causes many patients to wait for long periods with a progressively worsening condition.

Congestive heart failure is characterized by cardiac dilation or enlargement of the heart. In some cases, such as post-myocardial infarction or heart attack, the dilation may be localized to only a portion of the heart. In other cases, such as hypertrophic cardiomyopathy, there is typically increased resistance to filling the left ventricle producing dilation of the left atria. In dilated cardiomyopathy, the dilation is typically of the left ventricle with resultant failure of the heart as a pump. In advanced cases, dilated cardiomyopathy involves the majority of the heart. With each type of cardiac dilation, there are associated problems ranging from arrhythmias due stretching of the myocardial cells to leakage of the cardiac valves as a result of enlargement of the valvular annulus. As the heart enlarges, an increasing amount of work is required to pump the blood and, in time, the heart becomes so enlarged that it cannot adequately supply blood. A person afflicted with congestive heart disease feels fatigued, is unable to perform even simple exerting tasks, and experiences pain and discomfort.

Drug therapy is the most common treatment during the early stages of congestive heart disease. Drug therapy treats the symptoms of the disease and may slow the progression of the disease, but is not a cure for congestive heart disease. The disease will progress, even when treated with currently available drug therapy, and often the drugs produce adverse side effects.

Surgical procedures have been developed, or are under development, to treat heart dilation. These techniques include the Batista procedure, where a portion of the heart is dissected and removed in order to reduce heart volume. This is a radical and experimental procedure subject to substantial controversy. Like a heart transplant, the procedure is highly invasive, risky, expensive, and often includes other expensive procedures (such as a concurrent heart valve replacement). The treatment is limited to patients with the most severe levels of heart disease and, accordingly, provides little relief for patients with heart disease that is progressing toward its most serious stage following ineffective drug treatment. If the procedure fails, the only option currently available is an emergency heart transplant.

While there is a need for treatments, applicable to both early and later stages of congestive heart disease, that will either stop or more drastically slow the progress of the disease, there are few current treatment options. Cardiomyoplasty is a recently developed treatment for earlier stage congestive heart disease. In this procedure, the latisimus dorsi muscle (taken from the patient's shoulder) is wrapped around the heart and chronically paced synchronously with ventricular systole. Pacing of the muscle produces muscle contraction to assist the contraction of the heart during systole. Cardiomyoplasty has demonstrated symptomatic improvement but studies suggest the procedure only minimally improves cardiac performance. The procedure is highly invasive requiring harvesting a patient's muscle and an open chest (i.e., sternotomy) to access the heart. The cardiomyoplasty procedure is complicated. For example, it is difficult to wrap the muscle around the heart with a satisfactory fit and if adequate blood flow is not maintained to the wrapped muscle, the muscle may necrose. The muscle may stretch after wrapping reducing its constraining benefits and is generally not susceptible to postoperative adjustment. Finally, the muscle may fibrose and adhere to the heart causing undesirable constraint on the contraction of the heart during systole. The procedure is expensive and often requires a pacemaker to pace the muscle.

While symptomatic improvement may be accomplished with cardiomyoplasty, it has been suggested that some of the benefits derived from the procedure are the result of the external elastic constraint placed on the heart by the transplanted muscle. Alferness, U.S. Pat. No. 5,702,343, dated Dec. 30, 1997, discloses a device to constrain cardiac expansion during diastole. A cardiac constraint device, similar to a knit sock or jacket, is placed on an enlarged heart and fitted snug during diastole to limit expansion as the ventricle fills with blood. Care must be taken to avoid excessive tightening of the device and impairment of cardiac function. If the device is too tight, the left ventricle cannot adequately expand and left ventricular pressure will rise. While the constraint device reinforces the heart wall and impedes further enlargement of the heart, it does not provide assistance to a weakened heart muscle during systole.

Mechanical devices have been developed that assist the heart in pumping blood. These devices are used to treat congestive heart disease or, at least, provide a bridge to a heart transplant. Such devices include left ventricular assist devices (“LVAD”) and total artificial hearts (“TAH”). An LVAD typically includes a mechanical pump implanted under the diaphragm with tubes connected to the left ventricle and the aorta. The electrically or pneumatically powered pump urges blood flow from the left ventricle into the aorta assisting systole in a heart that has been weakened by heart disease. TAH devices are also used as temporary measures while a patient awaits a donor heart for transplant. These devices expose the patient to a risk of mechanical failure and frequently require external power supplies. The surgery to install an LVAD or TAH is expensive.

What is desired therefore, is a cardiac assist device that is of uncomplicated construction, resists further enlargement of the heart, and assists a weakened heart in supplying an adequate flow of blood.

### BRIEF DESCRIPTION OF THE DRAWINGS

FIG. 1 is a schematic cross-section of a normal healthy human heart during systole.

FIG. 1A is the view of FIG. 1 showing the heart during diastole.

FIG. 1B is a view of the left ventricle of a healthy heart as viewed from a septum and showing a mitral valve.

FIG. 2 is a schematic cross-section of a diseased human heart shown during systole.

FIG. 2A is the view of FIG. 2 showing the heart during diastole.

FIG. 2B is the view of FIG. 1B showing a diseased heart.

FIG. 3 is a side view of a cardiac assist device.

FIG. 3A is a side view of a diseased heart in diastole with the cardiac assist device of FIG. 3 in place.

FIG. 3B is a perspective view of the cardiac assist device of FIG. 3.

FIG. 4 is a side view of a second embodiment of a cardiac assist device.

FIG. 4A is a side elevation view of a diseased heart in diastole with the cardiac assist device of FIG. 4 in place.

FIG. 4B is a perspective view of the cardiac assist device of FIG. 4.

FIG. 5 is a schematic view of a cardiac assist device and a size adjusting inflatable bladder.

FIG. 6 is schematic view of a portion of a mesh cardiac assist device.

FIG. 7 is a side view of a third embodiment of a cardiac assist device.

FIG. 8A is an upper front perspective view of an electroactive polymer transducer.

FIG. 8B is an upper front perspective view of the electroactive polymer transducer of FIG. 8A in an actuated state.

FIG. 9 is a schematic of a cardiac assist system.

FIG. 10 illustrates an exemplary electrocardiogram trace.

FIG. 11 is a schematic illustration of a power supply arrangement for a cardiac assist system.

FIG. 12 is a schematic illustration of a polymer-metal composite transducer.

FIG. 13 is a front view of a cardiac assist device including polymer-metal transducers.

### DETAILED DESCRIPTION OF THE INVENTION

Congestive heart failure is characterized by cardiac dilation or enlargement of the heart. In some cases, such as post-myocardial infarction, the dilation may be localized to only a portion of the heart. In other cases, such as advanced dilated cardiomyopathy, the dilation involves the majority of the heart. With each level of cardiac dilation, there are associated problems ranging from arrhythmias to leakage of the cardiac valves due to enlargement of the valvular annulus. A person afflicted with congestive heart disease feels fatigued, is unable to perform even simple exerting tasks and experiences pain and discomfort. Congestive heart disease is progressive and, in time, the heart becomes so enlarged that it cannot adequately supply blood.

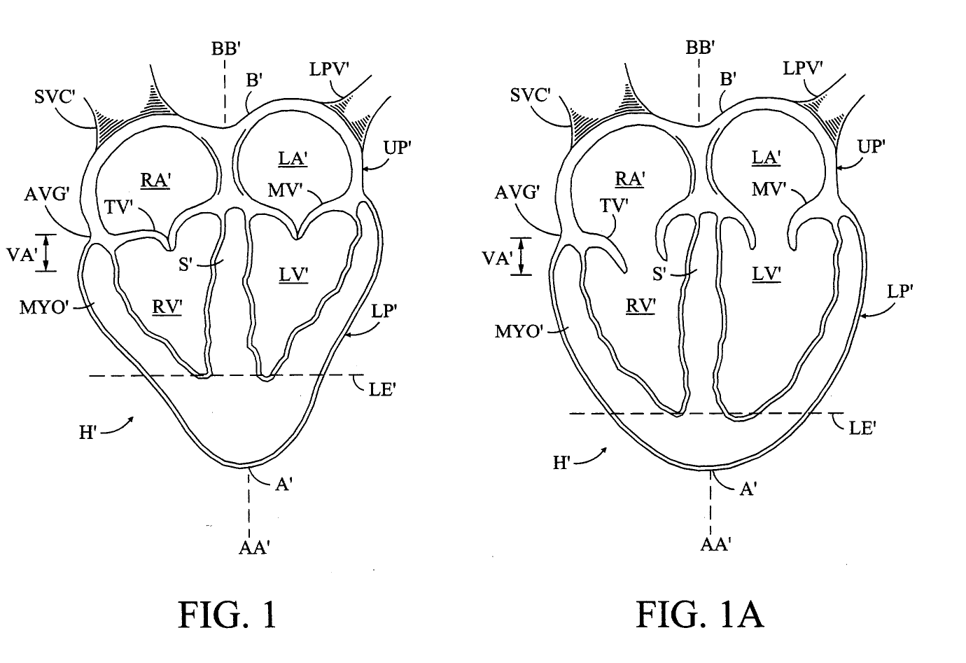

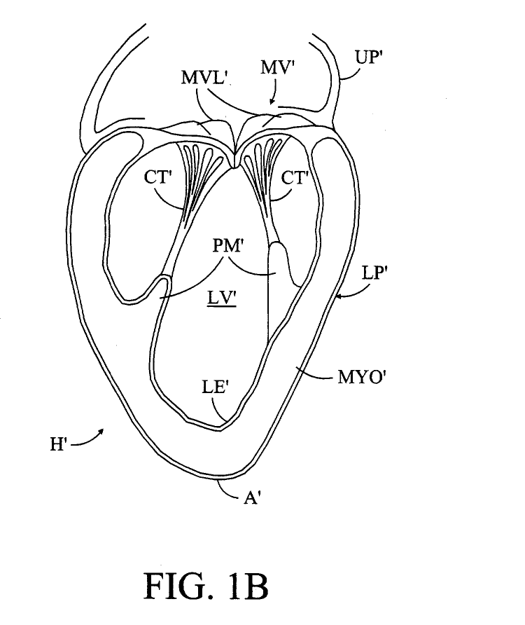

A normal, healthy human heart H′ is schematically illustrated, in cross-section, in FIGS. 1 and 1A. The heart H′ is a muscle having an outer wall or myocardium MYO′ and an internal wall or septum S′ . The myocardium MYO′ and septum S′ define four internal heart chambers including a right atrium RA′, a left atrium LA, a right ventricle RV′ and a left ventricle LV′. The heart H′ has a length measured along a longitudinal axis BB′-AA′ from an upper end or base B′ to a lower end or apex A′. The right and left atria RA′ and LA′ reside in an upper portion UP′ of the heart H′ adjacent the base B′. The right and left ventricles RV′ and LV′ reside in a lower portion LP′ of the heart H′ adjacent the apex A′. The ventricles RV′ and LV′ terminate at ventricular lower extremities LE′ adjacent the apex A′ and are spaced therefrom by the thickness of the myocardium MYO′. In FIG. 1, the heart H′ is shown during systole (i.e., high left ventricular pressure). In FIG. 1A, the heart H′ is shown during diastole (i.e., low left ventricular pressure).

Due to the compound curves of the upper and lower portions UP′ and LP′, the upper and lower portions UP′ and LP′ meet at a circumferential groove commonly referred to as the A-V (atrio-ventricular) groove AVG′. Extending away from the upper portion UP′ are a plurality of major blood vessels communicating with the chambers RA′, RV′, LA′ and LV′. For ease of illustration, only the superior vena cava SVC′, inferior vena cava IVC′ and a left pulmonary vein LPV′ are shown as being representative.

The heart H′ contains valves to regulate blood flow between the chambers RA′, RV′, LA′, and LV′ and between the chambers and the major vessels (e.g., the superior vena cava SVC′, inferior vena cava IVC′ and a left pulmonary vein LPV′). For ease of illustration, not all of such valves are shown. Instead, only the tricuspid valve TV′ between the right atrium RA′ and right ventricle RV′ and the mitral valve MV′ between the left atrium LA′ and left ventricle LV′ are shown as being representative. The valves are secured, in part, to the myocardium MYO′ in a region of the lower portion LP′ adjacent the A-V groove AVG′ and referred to as the valvular annulus VA′. The valves TV′ and MV′ open and close through the beating cycle of the heart H.

FIGS. 1 and 1A show a normal, healthy heart H′ during systole and diastole, respectively. During systole (FIG. 1), the myocardium MYO′ is contracting and the heart assumes a shape including a generally conical lower portion LP′. During diastole (FIG. 1A), the heart H′ is expanding and the conical shape of the lower portion LP′ bulges radially outwardly (relative to axis AA′-BB′). The motion of the heart H′ and the variation in the shape of the heart H′ during contraction and expansion is complex. The amount of motion varies considerably throughout the heart H′. The motion includes a component which is parallel to the axis AA′-BB′ (referred to as longitudinal- expansion or contraction) and a component perpendicular to the axis AA′-BB′ (referred to as circumferential expansion or contraction).

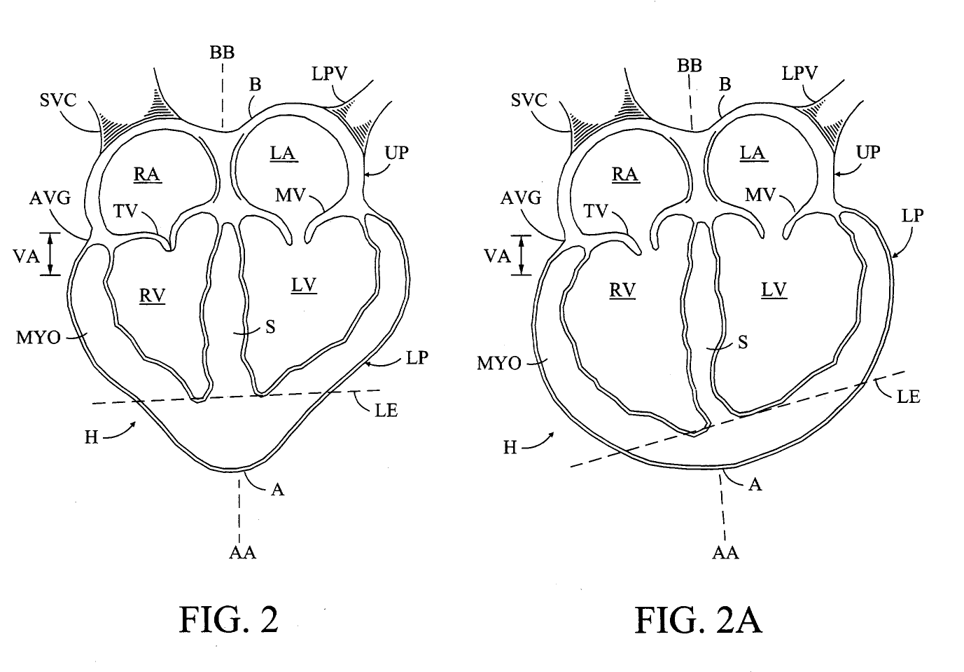

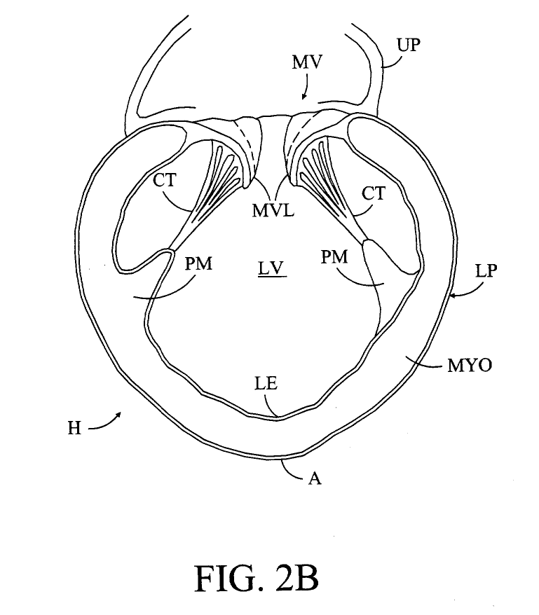

A heart deformed by congestive heart disease H is illustrated in systole in FIG. 2 and in diastole in FIG. 2A for comparison to the healthy heart H′ during, systole (FIG. 1) and diastole (FIG. 1A). All elements of the diseased heart H are labeled identically with similar elements of healthy heart H′ except for the omission of the apostrophe in order to distinguish the diseased heart H from the healthy heart H′.

Comparing FIGS. 1 and 2 (showing hearts H′ and H during systole), the lower portion LP of the diseased heart H has lost the tapered conical shape of the lower portion LP′ of the healthy heart H′. Instead, the lower portion LP of the diseased heart H dilates outwardly between the apex A and the A-V groove AVG. So deformed, the diseased heart H during systole (FIG. 2) resembles the healthy heart H′ during diastole (FIG. 1A). During diastole (FIG. 2A), the deformation is even more extreme.

While FIG. 2A indicates generalized deformation, localized heart dilation is often produced by myocardial infarction or heart attack. Myocardial infarction is the death of an area of the heart muscle due to a sudden loss of blood supply. The most common initiator of reduced cardiac blood supply is coronary atherosclerosis, a gradual build up of cholesterol plagues, scar tissue, and calcium deposits inside the coronary arteries. Once the opening in an artery has been narrowed, it is susceptible to sudden blockage by rupture of the cholesterol plagues or the formation of a blood clot in the damaged artery. A scar is left when the injured area of the muscle heals reducing the pumping efficiency of the heart. While in many cases there is sufficient good muscle left to provide an adequate blood supply, assistance for the damaged muscle may be required or desirable. Once infarction has occurred, the dying area of the heart muscle may disturb the normal sequences of electrical impulses that trigger operation of the heart muscle. Areas of the heart may begin to contract out of sequence, rather than pump rhythmically, further reducing the heart's own blood supply. These irregular rhythms can be fatal, even when sufficient muscle survives to pump an adequate supply of blood.

As a diseased heart H enlarges from the representation of FIGS. 1 and 1A to that of FIGS. 2 and 2A, the heart H becomes a progressively more inefficient pump requiring more energy to pump the same amount of blood. Continued progression of the disease results in the heart H being unable to supply adequate blood to the patient's body and the patient becomes symptomatic of cardiac insufficiency. The progression of congestive heart disease has been illustrated and described with reference to a progressive dilation of the lower portion LP of the heart H. While enlargement of the lower portion LP of the heart is most common and troublesome, enlargement of the upper portion UP may also occur.

In addition to cardiac insufficiency, the enlargement of the heart H can lead to valvular disorders. As the circumference of the valvular annulus VA increases, the leaflets of the valves TV and MV may spread apart. After a certain amount of enlargement, the spreading may be so severe the leaflets cannot completely close (as illustrated by the mitral valve MV in FIG. 2A). Incomplete closure results in valvular regurgitation contributing to an additional degradation in cardiac performance. While circumferential enlargement of the valvular annulus VA may contribute to valvular dysfunction as described, the separation of the valve leaflets is most commonly attributed to deformation of the geometry of the heart H. This is best described with reference to FIGS. 1B and 2B.

FIGS. 1B and 2B show a healthy and diseased heart, respectively, left ventricle LV′, LV during systole as viewed from the septum (not shown in FIGS. 1B and 2B). In a healthy heart H′, the leaflets MVL′ of the mitral valve MV′ are urged closed by left ventricular pressure. The papillary muscles PM′, PM are connected to the heart wall MYO′, MYO, near the lower ventricular extremities LE′, LE. The papillary muscles PM′, PM pull on the leaflets MVL′, MVL via connecting chordae tendineae CT′, CT. Pull of the leaflets by the papillary muscles functions to prevent valve leakage in the normal heart by holding the valve leaflets in a closed position during systole. In the significantly diseased heart H, the leaflets of the mitral valve may not close sufficiently to prevent regurgitation of blood from the ventricle LV to the atrium during systole.

As shown in FIG. 1B, the geometry of the healthy heart H′ is such that the myocardium MYO′, papillary muscles PM′ and chordae tendineae CT′ cooperate to permit the mitral valve MV′ to fully close. However, when the myocardium MYO bulges outwardly in the diseased heart H (FIG. 2B), the bulging results in displacement of the papillary muscles PM. This displacement acts to pull the leaflets MVL to a displaced position such that the mitral valve cannot fully close. While circumferential enlargement of the valvular annulus VA may contribute to valvular dysfunction as described, the separation of the valve leaflets is most commonly attributed to deformation of the geometry of the heart H.

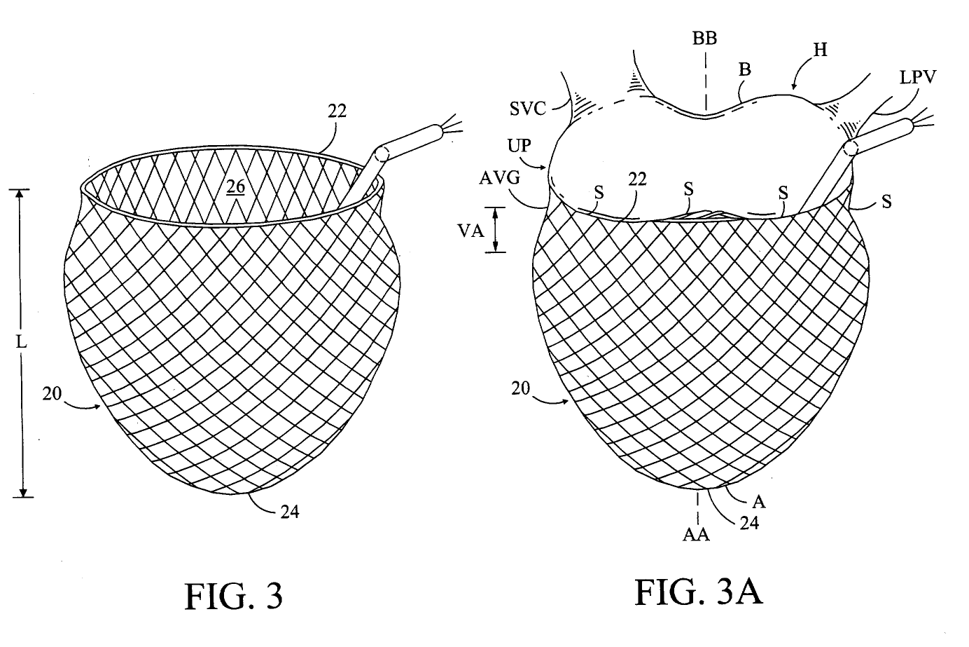

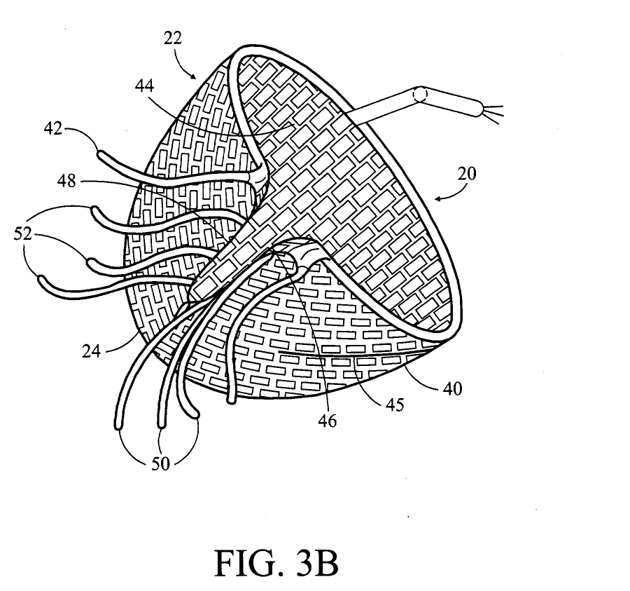

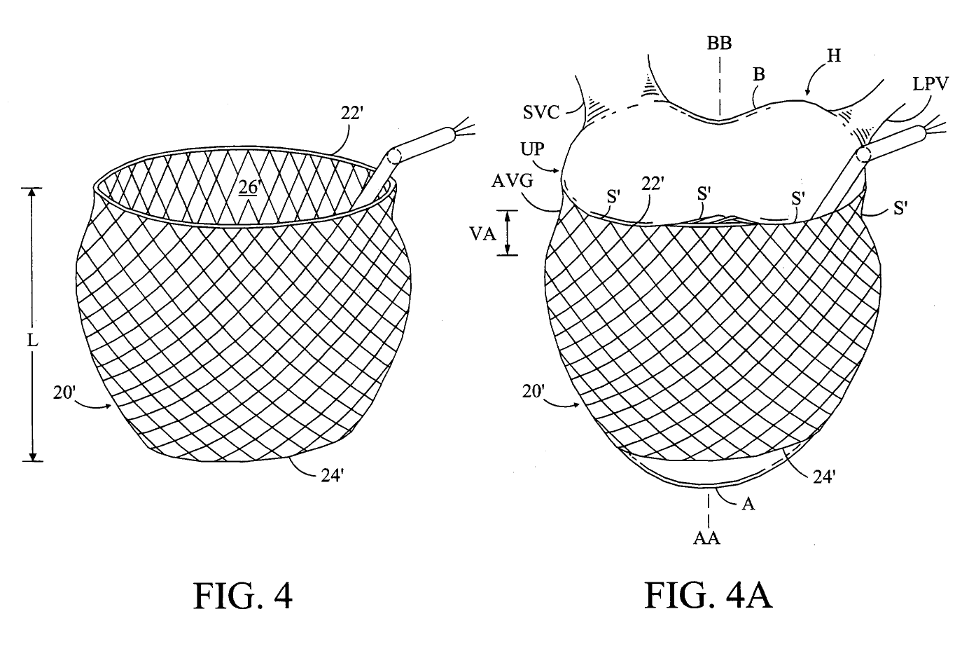

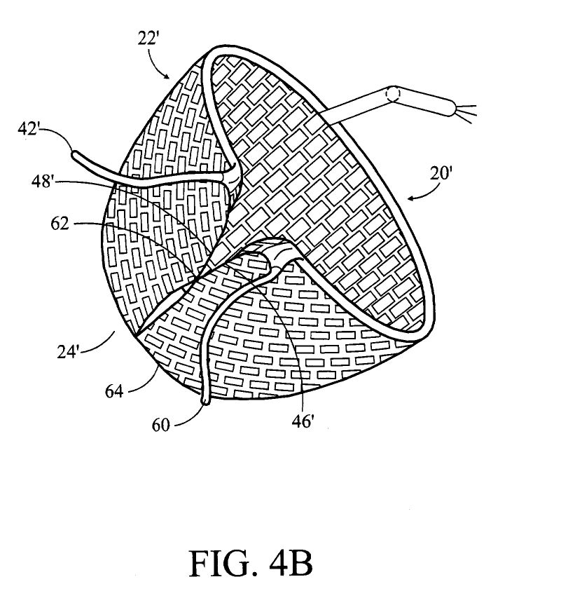

First and second embodiments of a cardiac assist device, jackets 20, 20′, are illustrated in FIGS. 3, 3A, 3B, 4, 4A, and 4B. The cardiac assist device fitted to and encircles a surface of the heart to limit the outward expansion of the heart wall during diastolic chamber filling and assist the contraction of the heart during systole. The jacket 20, 20′ comprises an enclosed cone-shaped tube having upper (base) and lower (apex) ends 22, 22′, 24, 24′. The jacket 20, 20′ defines an internal volume 26, 26′ which is completely enclosed but for the open ends 22, 22′ and 24′. In the embodiment illustrated in FIG. 3, the lower end 24 is closed and in the embodiment of FIG. 4, the lower end 24′ is open. In both embodiments, the upper ends 22, 22′ are open. Elements common to the embodiments illustrated in FIGS. 3 and 4 are numbered identically with the addition of an apostrophe to distinguish the second embodiment. Generally, the description herein refers to the embodiment illustrated in FIG. 3, and the common elements are not separately discussed.

As illustrated in FIGS. 3, 3A, and 3B, the jacket 20 is a mesh material 40, and includes a circumferential attachment device 42 at the base end 22 of the jacket. The apex end 24 of the jacket 20 is closed. The jacket 20 shown also includes a slot 44 having opposed lateral edges 46 and 48, and fasteners (e.g., lateral attachment device 50 and 52) for selectively adjusting the volumetric size of the jacket 20. The jacket 20 may also include radiopaque markers 45, such as radiopaque filaments, for visualizing the surface of the heart during radiographic study.

Similar to the embodiment illustrated in FIG. 3, the embodiment of FIGS. 4, 4A, and 4B includes a base end 22′ and an apex 24′ end. The base end includes a circumferential attachment device 42′ for securing the jacket 20′ to the heart H. The jacket 20′ also includes a slot 44′ having opposed lateral edges 46′, 48′. The lateral edges 46′, 48′ are shown pulled together at 60 by a lateral attachment device 62, for example, a suture. The embodiment shown in FIGS. 4, 4A, and 4B has an opening 64 at the apex end 24′ of the jacket 20′.

The jacket 20 is sized to fit the heart H during diastole. Typically, the physician determines the size of the jacket 20 to be applied to a particular heart based on cardiac performance or cardiac volume. The jacket 20 has a length L between the upper and lower ends 22, 24 sufficient for the jacket 20 to constrain the lower portion LP of the heart. The upper end 22 of the jacket 20 extends at least to the A-V groove AVG and further extends to the lower portion LP to constrain at least the lower ventricular extremities LE. The jacket 20 can be slipped around the heart H and the size adjusted by drawing the lateral edges 46 and 48 of the slot 44 together.

When the parietal pericardium is opened, the lower portion LP of the heart H is free of obstructions for applying the jacket 20 over the apex A. If, however, the parietal pericardium is intact, the diaphragmatic attachment to the parietal pericardium inhibits application of the jacket over the apex A of the heart. In this situation, the jacket can be opened along a line extending from the upper end 22′ to the lower end 24′ of jacket 20′. The jacket can then be applied around the pericardial surface of the heart and the opposing edges of the opened slot 44 secured together after placed on the heart. The opposing edges of the opened line can be drawn together to adjust the volume of the jacket and fastened to each other with one or more fasteners, such as a cord, suture, band, adhesive or shape memory element affixed to the edges. The lower end 24′ can then be secured to the diaphragm or associated tissues using, for example, sutures, staples, etc.

In the embodiment of FIGS. 3 and 3A, the lower end 24 of the jacket 20 is closed and the length L is sized for the apex A of the heart H to be received within the lower end 24 when the upper end 22 is placed at the A-V groove AVG. In the embodiment of FIGS. 4 and 4A, the lower end 24′ is open and the length L′ is sized for the apex A of the heart H to protrude beyond the lower end 24′ when the upper end 22′ is placed at the A-V groove AVG. The length L′ is sized so that the lower end 24′ extends beyond the lower ventricular extremities LE such that in both of jackets 20, 20′, the myocardium MYO surrounding the ventricles RV, LV is in direct opposition to material of the jacket 20, 20′ during diastole. Such placement is desirable for the jacket 20, 20′ to present a constraint against dilation of the ventricular portions of the heart H.

After the jacket 20 is positioned on the heart H as described above, the jacket 20 is secured to the heart. Preferably, the jacket 20 is secured to the heart H using sutures (or other fastening means such as staples). The jacket 20 is sutured to the heart H at suture locations S circumferentially spaced along the upper end 22. While a surgeon may elect to add additional suture locations to prevent shifting of the jacket 20 after placement, the number of such locations S is preferably limited so that the jacket 20 does not restrict contraction of the heart H during systole.

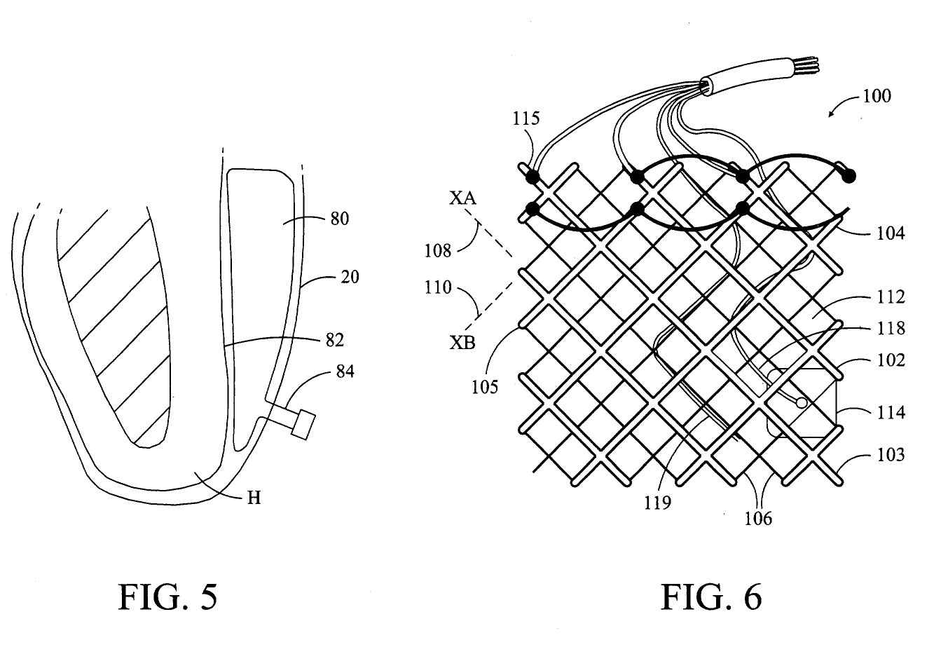

An alternative embodiment of an arrangement for selectively adjusting the size of a jacket 20 is illustrated in schematic cross-section in FIG. 5. According to this embodiment, an inflatable member 80 is inserted between the jacket 20 and the surface 82 of the heart H. The inflatable member 80 includes a filling apparatus 84 for entry of a fluid (liquid or gas) to inflate the inflatable member and reduce the volume of the jacket 20.

A cardiac reinforcement or constraint device aids the heart by reinforcing the heart wall and limiting the expansion of the heart during diastole. However, a cardiac reinforcement device does not assist the heart during systole. Assistance for the heart in pumping blood has heretofore been provided by a mechanical pump of a ventricular assist device (LVAD) or artificial heart. The present inventor realized that expansion of the heart during diastole could be limited and a weakened heart assisted during systole by a cardiac assistance system that included a contractile cardiac assist device to compress the heart, assisting the heart's natural contraction.

Referring to FIG. 6, compressive assistance to the heart H is provided by the cardiac assist device or jacket 20, 20′ which includes one or more electroactive polymer contractile transducers 102, 104 that are woven into the mesh fabric 100 of the jacket. A mesh 100 is schematically illustrated with fiber strands 106 and contractile transducers 102 and 104 interwoven on a plurality of axes XA 108 and XB 110 defining a diamond-shaped open cell 112. Filamentary transducers can be arranged along other axes to produce a mesh with triangular cells or cells of other shapes. A plurality of filaments in the mesh comprise one or more electroactive polymer contractile transducers 102, 104 that lengthen or shorten in response to the application of a voltage to the transducers' electrodes. As the transducers 102 and 104 are shortened or lengthened, the volume 26 of the jacket 20 is reduced or expanded, respectively, and the heart is compressed to aid the muscle in ejecting blood or decompressed to permit the ventricle to refill. The cardiac assist device 20 can be fitted to the heart and adjusted, post operatively, by permitting the contractile transducers 102, 104 to assume a length that produces an appropriate pressure during diastole. The blood pressure can be monitored by a pressure sensing transducer 117. For example, blood pressure may be sensed by a Doppler flow transducer. The Doppler flow transducer correlates blood velocity to a frequency shift in a sound reflected by blood in a vessel. The difference in frequency is proportional to the velocity of the blood which is correlated to blood pressure.

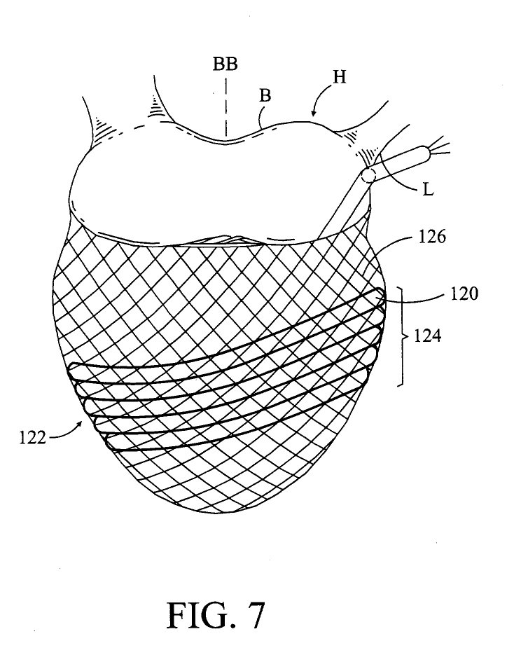

Referring to FIG. 7, in an alternative embodiment contractile transducers 120 of a cardiac assist device 122 are incorporated into a girdle 124 (indicated by a bracket) that encircles a surface of the heart H. As illustrated, the girdle 124 can be retained on the surface of the heart by a knit jacket 126 or sock of biomedical material.

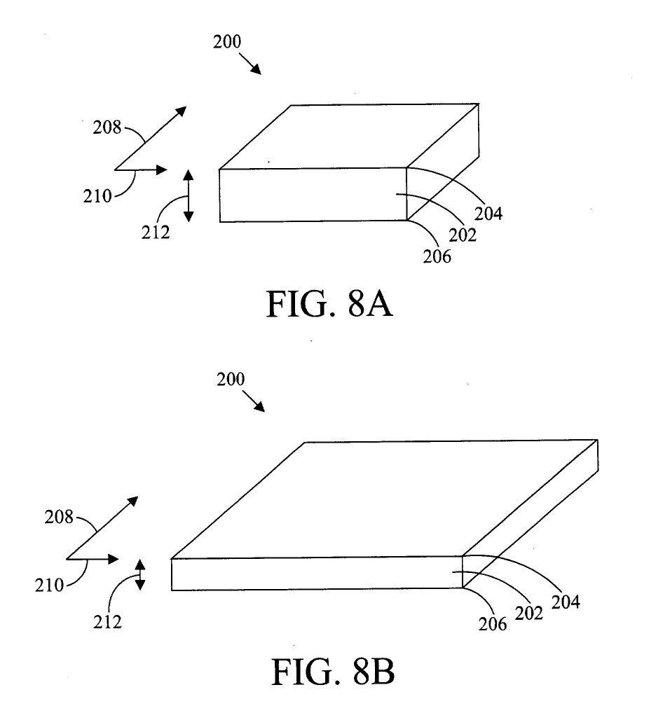

Electroactive polymers deflect when actuated by electrical energy. To help illustrate the performance of an electroactive polymer in converting electrical energy to mechanical energy, FIG. 8A illustrates a top perspective view of a transducer portion 200 comprising an electroactive polymer 202 for converting electrical energy to mechanical energy or vice versa. An electroactive polymer refers to a polymer that acts as an insulating dielectric between two electrodes and deflects upon application of a voltage difference between the two electrodes. Top and bottom electrodes 204 and 206 are attached to the electroactive polymer 202 on its top and bottom surfaces, respectively, to provide a voltage difference across a portion of the polymer. The polymer 202 deflects with a change in electric field provided by the top and bottom electrodes 204 and 206. Deflection of the transducer portion 202 in response to a change in the electric field is referred to as actuation. As the polymer 202 changes in size, the deflection may be used to produce mechanical work. In general, deflection refers to any displacement, expansion, contraction, torsion, linear or area strain, or any other deformation of a portion of the polymer. The change in the electric field corresponding to the voltage difference applied to or by the electrodes 204 and 206 produces mechanical pressure within the polymer 202. As illustrated by comparing the length 212, width 210, and depth 208 dimensions of FIGS. 8A and 8B electroactive polymer transducers deflect in all dimensions simultaneously. In general, the transducer portion 200 continues to deflect until mechanical forces balance the electrostatic forces driving the deflection. The mechanical forces include elastic restoring forces of the polymer material, the compliance of the electrodes 204 and 206, and any external resistance provided by a device or load coupled to the transducer element.

Electroactive polymers and electroactive polymer transducers are not limited to any particular shape, geometry, or type of deflection. For example, a polymer and associated electrodes may be formed into any geometry or shape including tubes and rolls, stretched polymers attached between multiple rigid structures, and stretched polymers attached across a frame of any geometry, including curved or complex geometries; or a frame having one or more joints. Deflection of electroactive polymer transducers includes linear expansion and compression in one or more directions, bending, and axial deflection when the polymer is rolled.

Materials suitable for use as an electroactive polymer may include any substantially insulating polymer or rubber (or combination thereof) that deforms in response to an electrostatic force or whose deformation results in a change in electric field. There are three primary types of electroactive polymers; ionic, molecular, and electronic. One suitable material is NuSil CF19-2186 as provided by NuSil Technology of Carpenteria, Calif. Other exemplary materials include silicone elastomers such as those provided by Dow Corning of Midland, Mich., acrylic elastomers such as VHB 4910 acrylic elastomer as produced by 3M Corporation of St. Paul, Minn., polyurethanes, thermoplastic elastomers, copolymers comprising PVDF, pressure-sensitive adhesives, fluoroelastomers, polymers comprising silicone and acrylic moieties, and the like. Polymers comprising silicone and acrylic moieties may include copolymers comprising silicone and acrylic moieties, polymer blends comprising a silicone elastomer and an acrylic elastomer, for example. Combinations of some of these materials may also be used as the electroactive polymer in transducers. The transducers 102, 104, 120 may be coated with a suitable biomedical material to avoid rejection or other unfavorable interaction with the body. Biomedical materials are materials that are physiologically inert to avoid rejection or other negative inflammatory response. Polyester, polytetrafluoroethylene (PTFE), expanded PTFE (ePTFE) and polypropylene are examples of biomedical materials.

When electrical power is applied to the contractile transducers 102, 104, 120, the transducers shorten in length compressing the heart and aiding the heart muscle in systole. The cardiac assist device 20 can include more than one contractile transducer 102, 104, 120. As illustrated in FIG. 6, a plurality of contractile transducers 102, 103 may be woven into the jacket 20 along one of a plurality of fiber axes XA 108 and a plurality of contractile transducers 104, 105 along another axis XB 110 of the plurality of fiber axes. The complex contraction of the heart can be mimicked by the cardiac assist device 20 by selective actuation of the various contractile transducers 102, 103, 104, 105 arranged along various axes in the mesh 100.

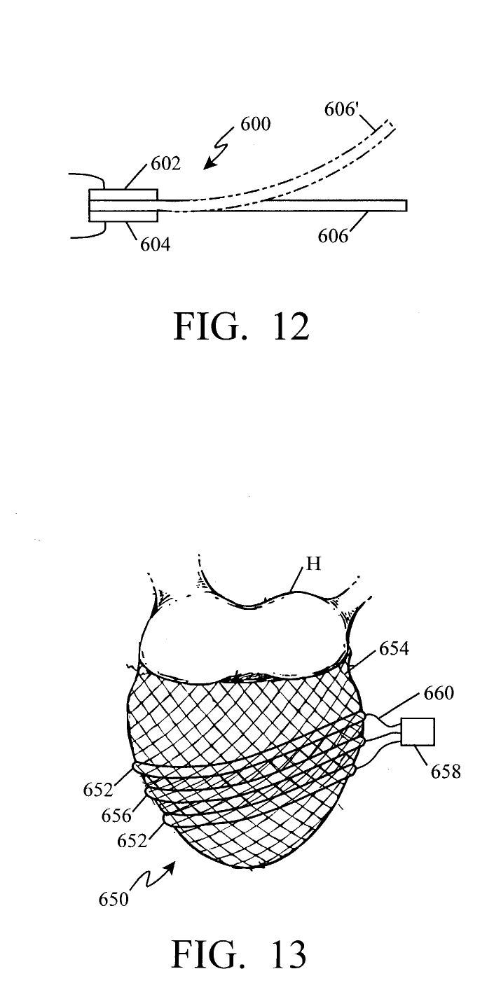

The contractile and sensing transducers of the cardiac assist device may comprise polymer-metal composite actuators and sensors. An ionic polymer-metal composite (IPMC) comprises a polymer having ion exchanging capability that is first chemically treated with an ionic salt solution of a conductive medium, such as a metal, and then chemically reduced. An ion exchange polymer refers to a polymer designed to selectively exchange ions of a single charge with its on incipient ions. Ion exchange polymers are typically polymers of fixed covalent ionic groups, such as perfluorinated alkenes, styrene-based, or divinylbenzene-based polymers. Referring to FIG. 12, a simple polymer-metal composite acutator or sensor 600 comprises suitable electrodes 602, 604 attached to a polymer-metal composite element. When a time varying electric field is applied to the electrodes 602, 604 attached a polymer-metal composite element 606, the element will exhibit a large dynamic deformation 606′. Referring to FIG. 13, an embodiment of the cardiac assist device 650 incorporates a plurality of polymer metal composite contractile transducers 652 for compressing the surface of the heart (H). The transducers 652 are restrained to the heart surface by a mesh basket 654. A voltage can be applied to the contractile transducers 652 of the cardiac assist device 650 through wires 660 connected to a plug 658 causing the transducers to deflect, compressing the surface of the heart (H).

On the other hand, when such a polymer-metal composite element 606 undergoes dynamic deformation, a dynamic electric field is produced across the electrodes 602, 604 attached to the composite element. A polymer-metal composite sensing transducer 656 is restrained to the mesh jacket 654 or the heart's surface so that when the jacket is deflected with the surface by the operation of the contractile transducers 652 and the heart's muscle the polymer-metal composite element 606 of the sensing transducer 656 is deflected producing a varying voltage at the electrodes of the sensing transducer that can be correlated to the transducer's deflection.

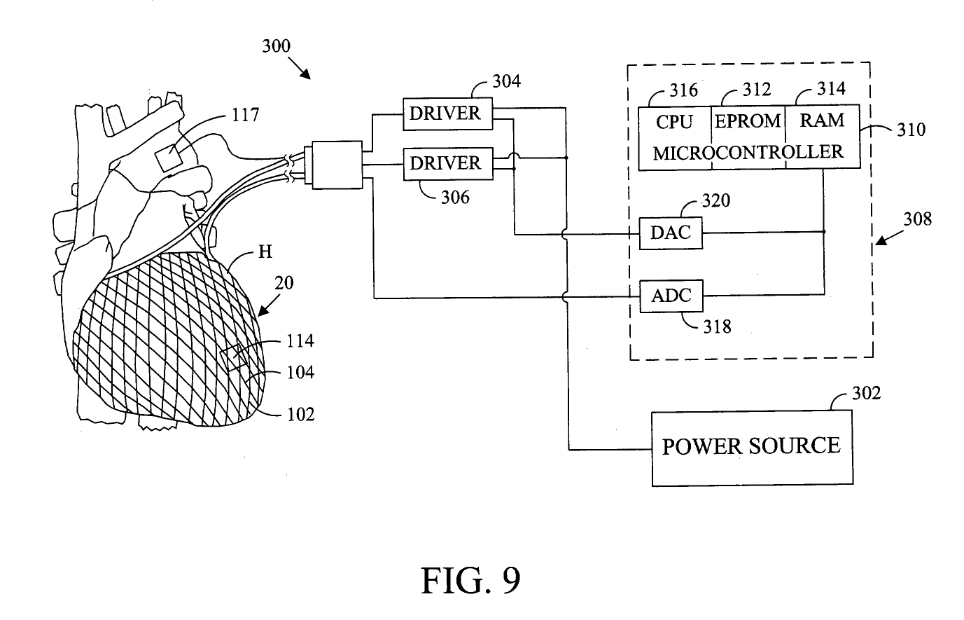

Referring to FIG. 9, an electroactive polymer transducer is actuated by connecting electrodes of the transducer to an electronic driver (for example, driver 304) that applies a voltage, from a power source 302, to electrodes in response to a control signal. A plurality of drivers 304, 306 can be used to control the actuation of a plurality of contractile transducers 102, 104.

Referring to FIG. 11, the power source 302 for the contractile transducers 102, 104 of the jacket 20 may be an internal power supply 502 that comprises an internal power source 503 and the drivers 304, 306 connected by appropriate leads 504 to the contractile transducers. The internal power source 503 may comprise a battery. The internal power supply 502 may comprise, in some embodiments, a radio frequency transducer for receiving and/or transmitting radio frequency signals to and from an external radio frequency (“RF”) transducer 506 which may send and/or receive RF signals to or from the internal power supply 502. Thus, the external RF transducer 506 may recharge a battery 503 within the internal power supply 502. Also, the external RF transducer 506 may be used to send signals to the drivers 304, 306 housed in the internal power supply 502 directing actuation of the contractile transducers 102, 104. In another embodiment, the controller 308 is housed in the internal power supply 502 and the external RF transducer 506 may be used to transmit program instructions and data regarding electromechanical sensing and/or cardiac parameters, such as pacing information, cardiac rhythm, degree of ventricular contraction, jacket tension, heart-rate information, or the like. Alternatively, the external RF transducer 506 may supply electrical power through inductive field coupling between the external RF transducer and the internal power supply 502.

In some embodiments, an external power source 508 can be used, which may be a battery pack. The external power supply 508 may supply current to the external RF transducer 506, which may in turn supply electrical energy to the internal power supply 502 through inductive field coupling. The technology for this inductive field coupling, including electronic programming and power transmission through RF inductive coupling, has been developed and is employed in, for example, cardiac pacemakers, automatic internal cardiac defibrillators, deep brain stimulators, and left ventricular assist devices.

The power requirements of the device of the disclosed embodiments is significantly lower than that of conventional LVAD because the heart continues to do some work while the contractile transducers 102, 104 of the jacket 20 augment native cardiac contractions.

Generally, the cardiac assist system 300 comprises the cardiac assist device or jacket 20 including one or more electroactive polymer contractile transducers 102, 104 to compress the heart H, and a controller 308 to generate appropriate signals to the drivers 304, 306 to actuate the contractile transducers according to a treatment regimen or in response to a cardiac parameter or characteristic sensed by a sensing transducer, for example transducer 114. In the cardiac assist system 300, the controller comprises generally, a microcontroller 310 including an erasable, programable, read-only memory (EPROM) 312 to store program instructions used to relate cardiac parameters, including requirements of a treatment regimen and sensed parameters, to output signals directing the contractile transducers 102, 104 in the cardiac assist jacket 20 to contract or extend; random access memory (RAM) 314 to store data and program instructions during processing; and a central processor (CPU) 316 to execute the program instructions and output signals directing action by the contractile transducers. The controller 308 typically includes an analog-to-digital convertor (ADC) 318 to convert analog signals output by the sensing transducers to digital data suitable for use by the microcontroller 310, and a digital-to-analog convertor (DAC) 320 to convert the digital output of the microcontroller to analog signals for operating the driver 304, 306 supplying power to the contractile transducers 102, 104.

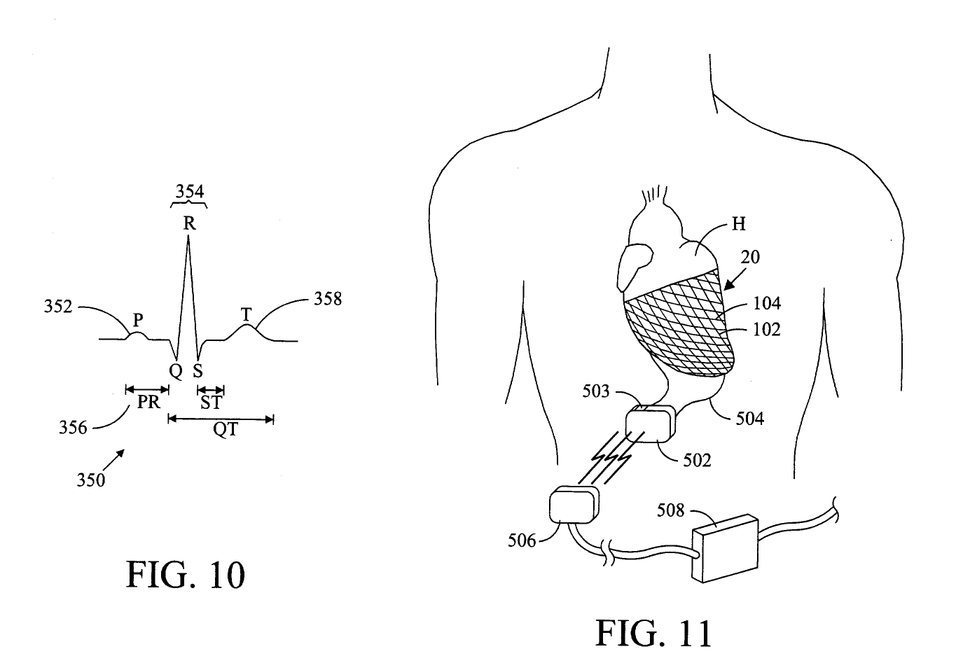

Generally, contraction of a contractile transducer 102, 104 is responsive to a signal generated by a sensing transducer 114 disposed in, around or near the heart. As the heart undergoes depolarization and repolarization, the electrical currents that are generated are detected by electrodes, such as sensing transducer 114, placed on the surface of the body or the heart. A pacemaker or pacer typically senses the electrical currents at the sino atrial (SA) and atrio ventricular (AV) nodes of the heart. Referring to FIG. 10, an electrocardiogram trace 350 represents the sequence of depolarization and repolarization of the heart's atria and ventricles. The P-wave 352 represents the wave of depolarization that spreads from the SA node throughout the atria initiating contraction of the atria musculature. The period between the onset of the P-wave and the initiation of the QRS complex 354 (indicated by a bracket) is termed the PR interval 358 and represents the time between the onset of atrial depolarization and the onset of ventricular depolarization. The QRS complex 354 represents ventricular depolarization causing myocyte contraction and an increase in the intraventricular pressure. When the intraventricular pressures exceed the pressures in the aorta and pulmonary artery, the aortic and pulmonic valves open and blood is ejected from the ventricles. Following ejection of the blood, ventricular repolarization is signaled by a T-wave 358. The ventricular muscle relaxes and the pressures in the ventricles fall causing the aortic and pulmonic valves to close. As the ventricular pressures drop below the artial pressures, the AV valves open and ventricular filling begins. Ventricular filling continues until the ventricles reach their full expansion causing the pressure in the ventricle to rise.

The onset of QRS 354 is detected by a sensing transducer 114, an electrode of the type used in a heart pacer to sense the electrical depolarization and repolarization signals of the heart. Examples of such electrodes include a ring electrode and a tip electrode. When the microcontroller 310 detects a particular signal from the sensing transducer 114, for example a signal indicating the onset of QRS 354, a program instruction causes the microcontroller 310 to output a signal to a driver 304, 306 to apply electrical power to one or more contractile transducers 102, 104 to compress the heart in rhythm with the natural muscular contraction. When the sensing transducer 114 inputs another signal to the microcontroller 310 indicating a change in the cardiac parameters, for example, the onset of the T 358-P 352 period, the microcontroller 310 signals a driver 304, 306 to interrupt or reverse the voltage applied to the electrodes of the contractile transducer 102, 104 relieving the pressure applied to the heart.

The electrode of the sensing transducer 114 may also be used to deliver pacing signals to the heart as part a cardiac rhythm management system. Pacers deliver timed sequences of low energy electrical stimuli, called pace pulses, to the heart, such as via an intravascular lead wire 119 or catheter (referred to as a “lead”) having one or more electrodes disposed in or about the heart. By properly timing the delivery of pace pulses, the heart can be induced to contract in proper rhythm, greatly improving its efficiency as a pump. Pacers are often used to treat patients with bradyarrhythmias, that is, hearts that beat too slowly, or irregularly.

The mesh material 100 is flexible to permit unrestricted movement of the heart H (other than uncontrolled expansion). The material is open defining a plurality of interstitial spaces for fluid permeability as well as minimizing the amount of surface area of direct contact between the heart H and the material of the jacket 20 (thereby minimizing areas of irritation or abrasion) to minimize fibrosis and scar tissue.

The open areas of the mesh 100 also allow electrical connection between the heart and surrounding tissue for passage of electrical current to and from the heart. For example, the open, flexible construction permits passage of electrical elements (e.g., pacer lead 114 or leads for ventricular cardioversion or defibrillation 119) through the assist device 20. Additionally, the open construction permits visibility of the heart's surface, thereby minimizing limitations to performing other procedures, e.g., coronary bypass, to be performed without removal of the jacket.

While the electrical signals generated by the heart H are conveniently used to control the actuation of the contractile transducers 102, 104, the sensing transducer 114 can be used to sense other heart parameters, such as blood pressure or motion of particular parts of the heart H, and program instructions can relate these parameters to output signals from the microcontroller 310 to actuate or modify the actuation of particular contractile transducers 102, 104. For example, a sensing transducer 115 comprising a piezoelectric material or an electroactive polymer in the jacket 20 may be used to sense the condition of the jacket and modify the operation of the contractile transducers 102, 104. The voltage between the electrodes of a piezoelectric material or electroactive polymer varies as the force applied (e.g., tension in a filament) to the sensing transducer 115 changes indicating the pressure being exerted by the jacket. For instance, the jacket 20 must not be too tight during diastole if the ventricle is to fill properly. However, some changes in the dimensions of the heart are healthy and accompany metabolic demands from physical exertion or exercise. An electroactive polymer sensing transducer 115 may be incorporated as one of the filaments of the mesh 100 to sense the tension in the interwoven filaments. If the tension exceeds a predetermined limit when diastole is signaled by the pace sensing transducer 114, the microcontroller 310 can signal the contractile transducer 102, 104 to relax the constriction applied by the transducer and relieve the pressure on the heart.

In summary, the jacket 20 constrains further undesirable circumferential enlargement of the heart while not impeding other motion of the heart H. The jacket assists the heart during systole by compressing the heart to aid the natural pumping action and relaxes during diastole to facilitate cardiac blood flow. The contractile transducers 102, 104 are triggered by signals from a pacer electrode that signaling the onset of the natural muscular contraction of the heart. The output of the sensing transducers, for examples, a pacer electrode and a pressure sensor, permit the control to sense the onset of cardiac arrest and initiate compression of the heart. The jacket 20 treats valvular disorders by constraining circumferential enlargement of the valvular annulus and deformation of the ventricular walls. The jacket 20 can be used in early stages and later stages of congestive heart disease.

The detailed description, above, sets forth numerous specific details to provide a thorough understanding of the present invention. However, those skilled in the art will appreciate that the present invention may be practiced without these specific details. In other instances, well known methods, procedures, components, and circuitry have not been described in detail to avoid obscuring the present invention.

All the references cited herein are incorporated by reference.

The terms and expressions that have been employed in the foregoing specification are used as terms of description and not of limitation, and there is no intention, in the use of such terms and expressions, of excluding equivalents of the features shown and described or portions thereof, it being recognized that the scope of the invention is defined and limited only by the claims that follow.

The invention claimed is:

1. A cardiac assist system comprising:
(a) a contractile transducer arranged to compress a surface of a heart in response to a first signal;
(b) a program for a data processing device including a program instruction; and
(c) a data processing device outputting said first signal to said contractile transducer in response to said program instruction.

2. The system of claim 1 wherein said contractile transducer comprises an electroactive polymer.

3. The system of claim 1 wherein said contractile transducer substantially encircles said surface of said heart and is of a size selected to constrain an expansion of said surface of said heart.

4. The system of claim 3 wherein said contractile transducer comprises an electroactive polymer interwoven with a substantially inelastic fiber.

5. The system of claim 3 wherein said contractile transducer comprises an electroactive polymer.

6. The system of claim 5 further comprising a knit jacket of substantially inelastic fibers retaining said contractile transducer to said surface of said heart.

7. The system of claim 1 wherein said contractile transducer comprises an electroactive polymer-metal composite.

8. The system of claim 7 further comprising a knit jacket of substantially inelastic fibers retaining said contractile transducer to said surface of said heart.

9. The system of claim 1 further comprising:
(a) a sensing transducer outputting a second signal to said data processing device, said sensing transducer being responsive to a condition of at least one of said heart and said contractile transducer; and
(b) a program instruction relating said second signal to said first signal.

10. The system of claim 9 wherein said sensing transducer responsive to at least one of a condition of at least one of said heart and said contractile transducer comprises an electrode sensing at least one of a depolarization and a repolarization of said heart.

11. The system of claim 9 wherein said sensing transducer responsive to at least one of a condition of at least one of said heart and said contractile transducer comprises a transducer sensing a tension in said contractile transducer.

12. The system of claim 11 wherein said sensing transducer comprises at least one of a piezoelectric material and an electroactive polymer.

13. The system of claim 9 wherein said sensing transducer responsive to at least one of a condition of at least one of said heart and said contractile transducer comprises a transducer sensing a flow of blood.

14. The system of claim 9 wherein said sensing transducer responsive to at least one of a condition of at least one of said heart and said contractile transducer comprises a transducer sensing a motion of said heart.

15. The system of claim 9 wherein said sensing transducer responsive to at least one of a condition of at least one of said heart and said contractile transducer comprises a transducer sensing a pressure.

16. The system of claim 11 wherein said sensing transducer comprises an electroactive polymer-metal composite.

17. A device for treating cardiac disease comprising an electroactive polymer contractile transducer arranged to compress a surface of a heart.

18. The device of claim 17 wherein said electroactive polymer contractile transducer substantially encircles said surface of said heart and is of a size selected to constrain an expansion of said surface of said heart.

19. The device of claim 18 wherein said electroactive polymer contractile transducer comprises an electroactive polymer interwoven in a mesh.

20. The device of claim 19 wherein said electroactive polymer is interwoven with a substantially inelastic fiber.

21. The device of claim 20 wherein at least one of said electroactive polymer and said substantially inelastic fiber comprises a biomedical material.

22. The device of claim 17 further comprising a jacket arranged to retain said contractile transducer to said surface of said heart, said jacket comprising a mesh of substantially inelastic fiber.

23. The device of claim 17 wherein said electroactive polymer contractile transducer includes
(a) a base end, said base end having an opening for applying said contractile transducer to said surface of said heart by passing said contractile transducer over said surface of said heart such that when applied to said surface, said base end of said contractile transducer is oriented toward a base of said heart; and
(b) a slot for selectively adjusting said size of said contractile transducer, said slot having opposing lateral edges which decrease said size of said contractile transducer by moving said opposing lateral edges together.

24. The device of claim 23 wherein said electroactive polymer contractile transducer substantially encircles said surface of said heart and is of a size selected to constrain an expansion of said surface of said heart.

25. The device of claim 24 wherein said electroactive polymer contractile transducer comprises an electroactive polymer filament of a mesh.

26. The device of claim 24 wherein said electroactive polymer contractile transducer is interwoven with a substantially inelastic fiber.

27. The device of claim 26 wherein at least one of said electroactive polymer and said substantially inelastic fiber comprises a biomedical material.

28. The device of claim 23 further comprising a jacket arranged to retain said contractile transducer to said surface of said heart, said jacket comprising an open mesh of substantially inelastic fiber.

29. The device of claim 24 wherein said electroactive polymer contractile transducer is interwoven with a substantially radiopaque filament.

30. The device of claim 23 further comprising an inflatable member mounted between said contractile transducer and said surface of said heart for selectively adjusting a size of said contractile transducer.

31. A device for treating cardiac disease comprising an electroactive polymer-metal composite contractile transducer arranged to compress a surface of a heart.

32. The device of claim 31 further comprising a jacket arranged to retain said contractile transducer to said surface of said heart, said jacket comprising a mesh of substantially inelastic fiber.
